(19) United States
(12) Patent Application Publication
(10) Pub. No.: US 2009/0104645 A1
(43) Pub. Date: Apr. 23, 2009

(54) Vectors And Methods For Identifying Proteins Amenable To Crystallization

(75) Inventors: James Eric Gouaux, Portland, OR (US); Toshimitsu Kawate, Portland, OR (US)

Correspondence Address: BRYAN CAVE LLP, 1290 AVENUE OF THE AMERICAS, NEW YORK, NY, 10104, US

(73) Assignee: The Trustees Of Columbia University In The City Of New York, New York, NY (US)

(21) Appl. No.: 12/187,911

(22) Filed: Aug. 7, 2008

Related U.S. Application Data
(63) Continuation of application No. PCT/US2006/004740, filed on Feb. 10, 2006.

(51) Int. Cl. C12Q 1/02

(52) U.S. Cl. 435/29

(57) ABSTRACT

The present invention provides for methods, systems and materials that may be used to identify and select proteins that are amenable to crystallization. While previous methods of screening proteins for crystallization required at least microgram quantities of purified protein, the inventive method can be practiced using nanogram quantities of unpurified protein.

### GRANT INFORMATION

The subject matter of this application was developed at least in part under National Institute of Health Grant No. R01NS38631, so that the United States Government holds certain rights herein.

### 1. INTRODUCTION

The present invention provides for methods, systems and materials that may be used to identify and select proteins that are amenable to crystallization. While previous methods of screening proteins for crystallization required at least microgram quantities of purified protein, the inventive method can be practiced using nanogram quantities of unpurified protein.

### 2. BACKGROUND OF THE INVENTION

X-ray crystallography is the most powerful technique to determine the atomic structure of biological macromolecules (Hendrickson, 2000). The resulting atomic structures, in turn, not only provide insight into mechanism but may also accelerate the discovery of therapeutic agents (Blundell et al., 2002; Kuhn et al., 2002). For example, the development of anti-human immunodeficiency virus (HIV) drugs for the treatment of AIDS benefited enormously from the atomic resolution structures of the water-soluble proteins HIV protease and HIV reverse transcriptase (Kaldor et al., 1997; Wlodawer and Vondrasek, 1998). However, most drugs currently prescribed target membrane proteins rather than water-soluble ones (Zambrowicz and Sands, 2003). Unfortunately, membrane proteins have proven more refractory to crystallization than water-soluble proteins, and thus there are relatively few atomic resolution structures of membrane proteins (Tate, 2001; Walian et al., 2004).

The P2X receptors are fast-acting eukaryotic ligand-gated ion channels expressed in membranes throughout the nervous system. P2X, along with the slow-acting G-protein coupled P2Y receptors, constitute the two types of purinergic ATP P2 receptors (Illes and Ribiero, 2004; North and Barnard, 1997). P2X receptors are opened by extracellular ATP or its structural analogs to generate Ca2+ influx which signals for proliferation, differentiation, cell-death, and nociception. Seven P2X subunits (P2X₁₋₇) have been found in different eukaryotes (Valera et al., 1994). Despite a number of studies by electrophysiology, biochemistry, and cell biology, the molecular mechanism of ligand-gating and channel activities are unknown due to a lack of structural information. Because adenosines like ATP play a role in a variety of biological aspects such as ischemia, hypoxia, anxiety, alcohol sensitivity, and dopaminergic neurotoxicity in Parkinson's diseases (Yaar et al., 2005), the P2X receptors may also play a role in mediating ATP's role in these aspects.

The current model shows that P2X receptors consist of trimers, or multimers of trimers, of P2X subunits (Aschrafi et al., 2004; Nicke et al., 1998; Nicke et al., 2003). Although the subunits can form both homomeric and heteromeric combinations, electophysiological evidence suggests that heteromeres dominate in vivo (Cook et al., 1997; Zhong et al., 1998). Because varying receptor subunit composition alters permeation properties and gating kinetics, the complexity in receptor assembly allows for diverse responses in ATP signaling. Each of the subunits is composed of two transmembrane domains that separate a large (277-289 residues) extracellular domain from the smaller intracellular n-terminus (˜20-30 residues), and c-terminus (28-242 residues). The ATP-binding site is predicted to be located in the extracellular domain, while the intracellular c-terminus can interact with serotonin (5-HT₃) and GABA type A receptors. Specifically, residue Lys 68 near the first transmembrane domain in P2X₁, and Pro 272 near the second transmembrane domain are believed to play an important role in binding ATP (Ennion et al., 2000; Jiang et al., 2000; Roberts and Evans, 2005). The Knowledge of the three dimensional architecture and atomic structure of P2X receptors is very limited because the crystal structure is not available, and there is no significant homology of P2X with any channel whose crystal structure has been solved.

Studies of mice deficient for various P2X receptors has shown that this receptor class is involved in a diverse array of physiological functions. Male mice lacking P2X₁ experience a ˜90% decrease in fertility that electrophysiological experiments suggest is due to a reduction in response to sympathetic nerve stimulation for vas deferens contraction. (Mulryan et al., 2000). Other studies have shown that the loss of P2X₁ reduces tubuloglomerular feedback response, suggesting that the receptor plays a role in renal autoregulation and renal blood flow (Inscho, et al., 2004). P2X₂ has been implicated in carotid body function, regulation of ventilatory responses to hypoxia (Rong et al., 2003), and the progression of fast synaptic excitation in myenteric S neurons (Ren et al., 2003). The loss of P2X₃ leads to enhanced thermal hyperalgesia in chronic inflammation, while behavioral responses to noxious mechanical stimuli are normal (Souslova et al., 2000). Moreover, P2X₃ knockout mice exhibit a marked urinary bladder hyporeflexia, suggesting that P2X₃ receptor mediated ATP-signaling is crucial for peripheral pain responses and afferent pathways controlling urinary bladder volume reflexes (Cockayne et al., 2000). Evidence also supports a role for P2X₇ in the production of interleukin-1β from peritoneal macrophages during the initiation of the inflammatory response's cytokine cascade (Lasbasi et al., 2002; Solle et al., 2001). Additionally, P2X₇ has been shown to be critical for the ATP-evoked release of GABA and glutamate in the mouse hippocampus (Papp et al., 2004).

Although mutational analysis of P2X₄₋₆ is lacking, expression of P2X₄ has been observed to increase in rat spinal microglia after nerve injury (Tsuda et al., 2003). Also, blocking P2X₄ with antagonists abolishes pain-sensing behaviors evoked by light touch to the paw, suggesting a role for P2X₄ in tactile allodynia and spinal microglia after nerve injury

P2X receptors are expressed throughout he nervous system and thought to govern fast synaptic transmission. The receptors are expressed in presynaptic dorsal root ganglia neurons, and elicit glutamate release upon ATP application (Gu and MacDermott, 1997). P2X mediated glutamate release has also been demonstrated in the rat spinal cord nociception pathway (Bardoni et al., 1997; Nakatsuka and Gu, 2001), while GABA release from glial cells in retina is increased upon P2X receptor activation (Neal et al., 1998).

Postsynaptic localization of P2X with GluR2/3 has also been observed in the cerebellum and the CA1 region of the hippocampus, suggesting an excitatory glutamatergic localization of the receptors (Rubio and Soto, 2001). Furthermore, ATP application in brain slices facilitates spontaneous excitatory and inhibitory postsynaptic currents that are blocked by P2X receptor antagonists, suggesting an involvement of postsynaptic P2X receptors in CNS neurotransmission (Watano et al., 2004).

In addition to the electrophysiological and biochemical studies of P2X receptors, solving the crystal structure would help to better discern the molecular mechanism of P2X ligand-gating and channel activities. To understand how one might increase the likelihood of obtaining crystals of membrane proteins, it is instructive to consider the typical flow of experiments involved in the crystallization of both water-soluble and membrane proteins.

In general, the probability of obtaining well-ordered crystals of a molecule is greater if the molecule adopts a single association state, i.e. it is monodisperse, if it is chemically and conformationally homogeneous, and if it is stable throughout the course of crystal growth. The likelihood of obtaining crystals can be increased if one examines variants of the target protein derived from the same parent protein, or derived from related proteins found in different organisms (Nishida and MacKinnon, 2002). The traditional approach to preparing target proteins for crystallization trials is summarized in FIG. 1A. Here, one typically begins by cloning a number of related genes and determining expression and purification conditions, using absorbance at 280 nm to detect and quantify the target proteins.

Following purification, one may then evaluate the homogeneity and stability of the protein—referred to here as precrystallization screening. One of the most useful tools to monitor the monodispersity and stability of the target protein is size-exclusion chromatography (SEC); a monodisperse and stable protein will yield a single, symmetrical Gaussian-shaped peak, while a polydisperse, unstable protein will give multiple, asymmetric peaks. Because the presence of the target protein using conventional methods is typically followed by absorbance at 280 nm, microgram to milligram quantities of the target protein are required for reliable detection. Furthermore, because almost all proteins, as well as nucleic acids, absorb at 280 nm, the target protein must be free of major contaminants. Thus, a substantial investment in time and resources must be made in order to bring a target molecule from the cloning stage to precrystallization screening. Moreover, because many target molecules fail precrystallization screening, due to polydispersity or instability, the time and resources invested in moderate to large-scale expression and purification are frequently wasted.

Formation of well-ordered crystals is a bottleneck for structure determination of membrane proteins by x-ray crystallography. Nevertheless, one can increase the probability of successful crystallization by precrystallization screening, a process by which one analyzes the monodispersity and stability of the protein-detergent complex. Traditionally, this has required microgram to milligram quantities of purified protein and a concomitant investment of time and resources. A precrystallization screening strategy that allows for the screening of small quantities f unpurified proteins would decrease the time and resources invested in determining the suitability of a candidate target protein for crystallization, and increase the number of target proteins that can be screened.

### 3. SUMMARY OF THE INVENTION

The present invention relates, at least in part, to the discovery that the proclivity of a cloned test protein to crystallize may be evaluated by forming a fusion protein between the test protein and a fluorescent protein (“FP”) to form a fluorescent test protein, or “FTP,” performing size exclusion chromatography on solubilized FTP, detecting the fluorescence of eluent fractions containing FTP to determine the FTP's elution profile, and then characterizing the elution profile. An elution profile characterized by a substantially symmetrical, single peak indicates that the FTP is monodisperse and therefore likely to crystallize easily. In contrast, where the FTP profile comprises several peaks, or an asymmetric peak, the test protein is unlikely to readily crystallize. Moreover, where the test protein elutes in the void volume, it is probably unstable, and therefore unlikely to easily form crystals.

According to the present invention, the solubilized FTP need not be purified, and may be present in the context of an unpurified or partially purified cell lysate. Moreover, nanogram quantities of FTP may be sufficient for testing. This is a substantial advantage relative to the prior art, which requires microgram or milligram quantities of purified test protein.

The present invention therefore provides a more time and materials-efficient method for identifying proteins amenable to crystallization. Using the present invention, the skilled artisan is enabled to screen larger numbers of proteins, and more quickly identify proteins for further study. As the determination of crystal structure facilitates rational drug design and permits virtual screening to identify molecules that bind to and/or have a functional relationship with a test protein, the present invention provides an important component in the drug discovery process. The present invention is particularly advantageous as applied to the screening of membrane proteins, which have special biological significance as mediators between extracellular and intracellular environments, and yet have been known in the art to be notoriously difficult to crystallize. In working examples herein, the effectiveness of the present invention in screening membrane proteins for crystallizability is demonstrated.

### 4. BRIEF DESCRIPTION OF THE FIGURES

FIG. 1A-B. Flow Chart of Precrystallization Screening. (A) Traditional screening. Variants of a target protein are expressed and purified on mid to large scales to produce microgram to milligram quantity of samples. The resulting purified proteins are characterized for monodispersity and stability by a series of biochemical assays. This precrystallization screening is continued until a promising construct is identified, often which is subjected to crystallization trials. (B) FSEC screening. Variants of a target protein are expressed as GFP fusions on a small scale and characterized directly by FSEC without purification. Here, the fusion proteins are analyzed for expression level, monodispersity, approximate molecular mass, and stability. Once a promising construct is determined, it is expressed and purified on a mid to large scale to screen crystallization conditions.

FIG. 2A-D. Maps of the GFP-fusion Vectors. (A) Eukaryotic expression vector pCGFP-EU. (B) Bacterial expression vector pCGFP-BC. (C) Eukaryotic expression vector pNGFP-EU. (D) Bacterial expression vector pNGFP-BC. In vectors pCGFP-EU. and pNGFP-EU, transcription is driven by a cytomegalovirus promoter (CMV) and terminated by SV40 polyadenylation sequences, and sequences encoding a polyhistidine tag, a thrombin proteolysis site and enhanced green fluorescent protein (EGFP) are located at either the 5′ or 3′ end of the multiple cloning sites (MCS). For the bacterial expression vectors pCGFP-BC and pNGFP-BC, transcription is directed by a T7 promoter and terminated by a T7 terminator sequence. The coding sequence is designed in the same way as the eukcaryotic expression vectors, except that a variant of uvGFP, which has codons optimized for bacterial expression, is used instead of EGFP.

FIG. 3A-B. Flow Chart of Precrystallization Screening using GFP-fusion Proteins and FSEC. (A) A gene of interest is amplified by PCR and subcloned into one of the GFP-fusion vectors. Cells are either transfected or transformed with the expression vectors and solubilized with a detergent containing buffer. (B) The resulting crude cell lysate, following centrifugation, is directly loaded onto a SEC column. The SEC column eluent is then passed through a flow-cell in a fluorometer set to detect GFP fluorescence. In this schematic, the FSEC setup includes a UV detector and a fraction collector, elements which are useful for running standards or for purifying a fusion protein based on its fluorescence profile. The panel labeled “fluorescence” is a hypothetical elution profile of a GFP-fusion protein detected by GFP fluorescence. The panel labeled “UV absorbance” represents a model of typical UV absorbance pattern from a crude cell lysate.

FIG. 4A-C. Subtype Screening of P2X Receptors by FSEC. (A) Fluorescence microscopic images of HEK293 cells expressing P2X-EGFP fusion proteins. The images were taken 48 hours after transfection. The scale bar is 100 nm. (B) FSEC traces from C-terminally tagged P2X1-5, 7 (C-P2X1-5, 7). (C) FSEC traces from N-terminally tagged P2X3-5 (N-P2X3-5) including C-P2X3 and C-P2X5. The arrows indicate the estimated elution position of the void volume, a P2X oligomer (ca. trimer to hexamer), a monomeric P2X subunit, and free EGFP, respectively. Note the difference in scales for the top and bottom panels.

FIG. 5A-F. Screening Detergents by FSEC. FSEC analysis of protein J in 6 different detergents: (A) C₁₂M, (B) C₁₀M, (C) β-OG, (D) C₁₂E₈, (E) LDAO and (F) CHAPS. The arrows indicate the estimated elution position of the void volume, an oligomer species (ca. trimer-hexamer), and free EGFP. The substantial peak due to free GFP may arise from either proteolysis of the fusion protein or from translation initiation at the methionine residue at the beginning of the GFP coding sequence.

FIG. 6A-F. Crystallization of Membrane Proteins Screened by FSEC (A) FSEC traces from C-terminally tagged bacterial membrane proteins. Six different genes (1-6) were screened using pCGFP-BC expression vector. (B) Rod-shaped crystals of gene #2 protein are shown. The length of the bar is 200 μm. (C) A diffraction image from a rod-shaped crystal that diffracted beyond 2.8 Å resolution. (D) FSEC traces from either N- or C-terminally tagged an eukaryotic glutamate transporter homologue from P. horikoshii (GltPh). The transporter gene was expressed with pCGFP-BC or pNGFP-BC, and the behavior of these fusion proteins were examined by FSEC. (E) Hexagonal crystals of GltPh protein are shown. The bar indicates 200 μm. (F) A diffraction image from the hexagonal crystal. These crystals diffract anisotropically to ca. 3.2 Å along c* and 3.8 Å along a*.

FIG. 7A-C. Gaussian Peak Fitting of FSEC Trace. Fitting of FSEC traces of (A) C-Glt_{Ph} and (B) N-Glt_{Ph} with Gaussian functions. A summary of the peak fitting parameters is shown in (C). The solid lines indicate the separate Gaussian functions and the dashed lines represent the sum of the individual Gaussians.

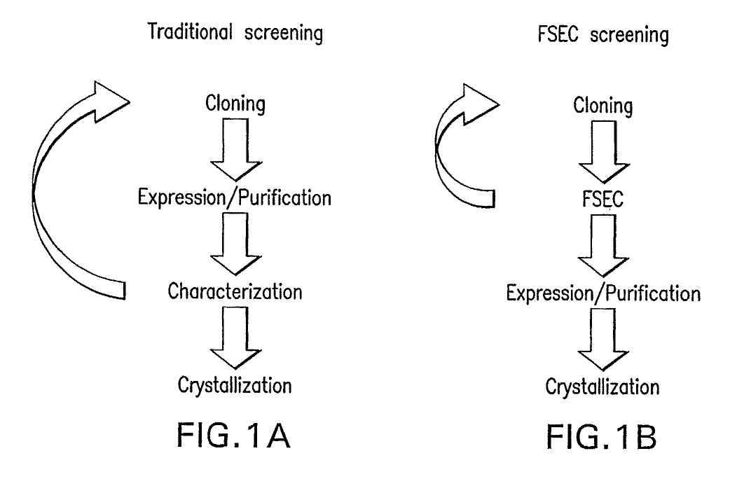

### 5. DETAILED DESCRIPTION OF THE INVENTION

For clarity, and not by way of limitation, the detailed description of the invention is divided into the following subsections:

(i) constructs for expressing FTPs;

(ii) fluorescence detection size exclusion chromatography;

(iii) elution profile assessment;

(iv) detergent screening; and

(v) uses of the invention.

5.1 Constructs for Expressing FTPs

The present invention provides for expression constructs and vectors comprising said constructs, for use in the methods of the invention. An expression construct of the invention comprises a nucleic acid encoding a fusion protein comprising a test protein and a fluorescent protein, operably linked to a promoter element.

A “test protein” is any protein that is being evaluated according to the invention, and may be any protein. In preferred non-limiting embodiments of the invention, the test protein may be one or more of the following: a protein intrinsic to the plasma membrane, a protein intrinsic to the nuclear membrane, a protein intrinsic to the endoplasmic reticulum membrane, a protein intrinsic to the mitochondrial membrane, a kinase, an ion channel protein, a receptor protein, or a transporter protein. Proteins for use as test proteins may be identified, for example, via the Gene Ontology website at http://www.geneontology.org/ and/or the AmiGO browser, or via the Entrez Protein Database at http://www.ncbi.nlm.nih.gov/entrez/query.fcgi?db=Protein.

A “fluorescent protein” (“TP”) as that term is used herein, is any protein that is intrinsically fluorescent, (including naturally occurring fluorescent proteins (such as Green Fluorescent Protein (“GFP”) from the jellyfish Aequorea Victoria), variants or portions thereof. Non-limiting examples of fluorescent proteins that may be used, according to the invention, include GFP of A. Victoria and fluorescent variants thereof (e.g., S65T, EGFP, GFPuv (see section 6, below)), FPs known in the art as “cyan FPs” (“CFPs”), “yellow FPs” (“YFPs”, including “YFP Venus” (Nagai et al., 2002, Nature Biotechnol. 20:87-90)), “blue FPs” (“BFPs”), and “red FPs” (“RFPs”) (quotations employed because color designation may be subjective or condition dependent), circularly permuted FPs (Baird et al., 1999, Proc. Natl. Acad. Sci. U.S.A. 96:11241-11246), monomeric RFPs (e.g., see Campbell et al., 2002, Proc. Natl. Acad. Sci. U.S.A. 99:7877-7882 and Bevis and Glick, 2002, Nature Biotechnol. 20:83-87); pH sensitive FPs (e.g., pH sensitive GFP (“pHluorin”); Meisenböck et al., 1998, Nature 394:192-195), photoactivatable FPs (e.g., photoactivatable GFP (Patterson et al., 2002, Science 297:1873-1877), voltage sensitive FPs (e.g., “FlaSh” (Guerrero et al., 2002, Biophys. J. 83:3607-3618) and “SPARC” (Ataka et al., 2002, Biophys. J. 82:509-516) and FPs from marine coelenterates, including but not limited to Renilla mulleri, Heteractis crispa, Entacmaea quadricolor, Discosoma and Trachyphyllia geoffroyi (for additional references, see Zhang et al., 2002, Nat. Rev. Mol. Bio. 3:906-918, Sawano et al., 2000, Nucl. Acids Res. 28:E78; Griesbeck et al., 2001, J. Biol. Chem. 276:29188-29194; Nagai et al., 2002, Nature Biotechnol. 20:87-90; Scholz et al., 2000, Eur. J. Biochem. 267:1565-1570; Baird et al., 1999, Proc. Natl. Acad. Sci. U.S.A.; Deitrich and Maiss, 2002, Biotechniques 32: 286, 288-90, 292-3; Su et al., 2001, Biochem. Biophys. Res. Commun. 287(2):359-65). In specific non-limiting embodiments, the present invention relates to fluorescent proteins having an amino acid sequence accessible in GenBank, including GFP (GenBank Acc. No. P42212), GenBank Accession Numbers: 1G7KA, 1G7 KB, 1G7KC, and 1G7 KD (for four chains of RFP of Discosoma); AAC53684 (a GFP); AA048591 (a YFP); YP 008577 (a BFP); and CAD53293 (a CFP). Fluorescent portions of, or variants of, such proteins may also be used.

The fluorescent protein may be fused to either the C-terminus or the N-terminus of the test protein. According to the present invention, a short stretch of amino acid residues (e.g., up to 5, 10, 15 or 20 residues (which means up to 5, up to 10, up to 15 or up to 20 residues)) of the test protein and/or the fluorescent protein may be omitted from the FTP, and may optionally be replaced by an artificial linker sequence. Accordingly, a “C-terminal portion” of the test protein or the fluorescent protein is construed to mean the C-terminus of the protein with up to 20 residues (preferably, up to 5, 10 or 15 residues) missing, and the “N-terminal portion” of the test protein or the fluorescent protein is construed to mean the N-terminus of the protein with up to 20 residues (preferably up to 5, 10 or 15 residues) missing, and the junction between the fluorescent protein and the test protein may optionally comprise a linker molecule of up to 5, 10, 15 or 20 residues. The linker molecule is an artificial sequence, in that it is not identical to the native terminal sequences of either the test protein or the fluorescent protein. Any deletion of amino acid residues at a terminus of the fluorescent protein does not abolish its fluorescence, except where fluorescence is restored by a linker molecule.

In preferred, nonlimiting embodiments, the expression construct further encodes an affinity tag, that may be used, in conjunction with affinity chromatography, to purify the fusion protein or fluorescent protein cleaved therefrom. Suitable non-limiting examples of affinity tags that may be encoded by the expression construct include a polyhistidine tag (see Section 6, below), FLAG tag, Myc tag, Hemagglutinin tag, Thioredoxin tag, V5 tag, Glutathione-S-Transferase tag, Maltose Binding Protein tag, Green Fluorescent protein tag etc.

In further preferred, non-limiting embodiments, the expression construct further encodes an enzyme cleavage site, placed in the fusion protein between the fluorescent protein and the test protein. Such an enzyme cleavage site may be provided by an artificial linker molecule. In specific non-limiting embodiments, an enzyme cleavage site and an affinity tag are located at opposite ends to the fluorescent protein in the fusion protein construct, thereby permitting of cleavage and removal of fluorescent protein. Suitable cleavage sites include, but are not limited to, a thrombin site (see Section 6 below), Factor Xa site, Intein site, PreScission Protease™ site etc.

The expression construct may be expressed in a bacterial host cell or a eukaryotic host cell. Suitable vectors and promoter elements may be selected based upon whether the construct is to be expressed in a bacterial or eukaryotic system.

Suitable promoters for expression in bacteria include those derived from bacterial or phage genes including T7 promoter, SP6 promoter, lac promoter, tac promoter, trc promoter, phage lambda P_{L} promoter, uspA promoter, uspB promoter, lacUV5 promoter, malK promoter, araB promoter etc.

Suitable promoters for expression in eukaryotic cells include the cytomegalovirus immediate early gene (“CMV”) promoter, SV-40 T-antigen promoter enhancer, Rous Sarcoma Virus LTR promoter, retroviral LTR-based promoters, human actin gene promoter, herpes simplex virus thymidine kinase promoter, adenoviral E1B gene promoter, human EF-1 alpha promoter, human metallothionine promoter etc.

Suitable vectors for expression in bacteria include the pGEX vector series for glutathione fusions (Amersham Biosciences, Piscataway, N.J.), pQE-vector series for Histidine fusions (Qiagen, Valencia, Calif.), pET and pRSET-vector series for Histidine fusions (InVitrogen, Carlsbad, Calif.), pBAD vector series (InVitrogen, Carlsbad, Calif.) etc.

Suitable vectors for expression in eukaryotic cells include the pCDNA vector series, pCEP and pREP vector series, pEF vector series, pZEO SV vector series for expression in mammalian cells; pBlueBac4.5 and pBlueBac4.5/V5-His and similar vectors for Baculoviral expression; pMT/BioEase or pAc5.1/V5-His or similar vectors for Drosophila expression; pHIL-D2 and pPIC3.5 vectors for intracellular expression and pHIL-S1 and pPIC9 vectors or similar plasmids for secreted expression in Pichia pastorus expression systems; the pYES2.1-E or related vectors for yeast expression; adenoviral expression vectors such as the pAd/CMV/V5-DEST™ or related vectors; lentiviral expression vectors such as pLenti6/V5-DEST™, and pLenti4/V5-DEST™, pLenti6/UbC/V5-DEST™ etc.

5.2 Fluorescence Detection Size Exclusion Chromatography

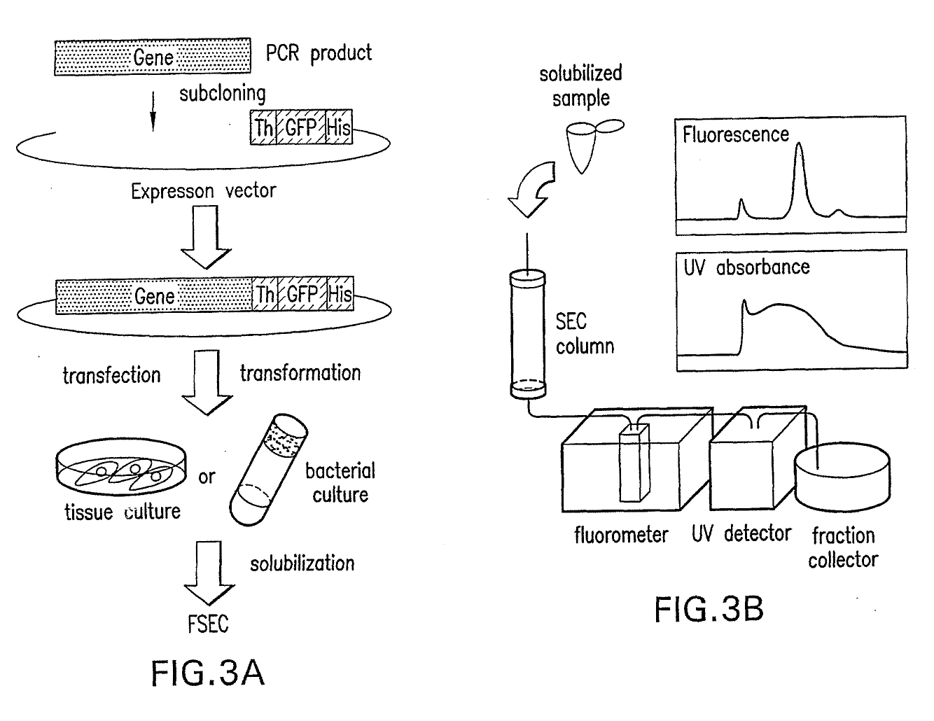

The present invention provides for a system for performing pre-crystallization screening of proteins comprising a size exclusion chromatography apparatus comprising a size exclusion chromatography column (“SEC”), coupled to a fluorimeter fitted with a flow cell, whereby eluant from the SEC passes through a flow cell, permitting the fluorescence of successively eluting materials, at a specified excitation and emission wavelength, to be read and preferably stored in electronic or tangible (e.g. paper) form. In preferred non-limiting embodiments, the eluent also passes through an ultraviolet wavelength (“UV”) detector either prior to or following passage through the fluorimeter. In further preferred non-limiting embodiments, the eluent also passes through a fraction collector. The fraction collector may be used to produce eluent fractions corresponding to the elution profile of a FTP. A schematic drawing of one non-limiting embodiment of the invention is shown in FIG. 3B.

The UV detector may be used to calibrate the system using standard proteins. For example, an EGFP standard may be applied to the SEC, and its fluorescence profile (at an excitation wavelength of 455 nm and an emission wavelength of 507 nm) and UV profile (at 280 nm) may be correlated. The incorporation of a UV detector in the system does not presume that FTP would be present in UV detectable amounts.

The excitation and emission wavelengths vary between fluorescent proteins, so that the excitation and emission wavelengths utilized by a fluorimeter are determined by the particular fluorescent protein used. For EGFP, as set forth above, appropriate excitation and emission wavelengths are 488 nm and 507 nm, respectively. For GFPuv, an excitation wavelength of 395 nm and an emission wavelength of 507 nm may be used (see Section 6, below).

Likewise, the flow rate, time increment, integration time and recordation time (see Section 6, below) may be varied according to experimental conditions and to obtain optimized peal resolution.

SEC is otherwise performed using techniques known and materials known in the art.

FTP for analysis by the above system may be applied to the SEC column in unpurified, partially purified, or purified form. In one preferred non-limiting embodiment, an FTP-containing sample for application to the SEC column may be prepared by introducing an FTP-encoding expression construct into a eukaryotic host cell using transient transfection techniques, culturing the transfected cell for between about 24 and 48 hours to produce a population of cells expressing FTP, and then lysing said cells with a solubilization buffer comprising a detergent (and preferably a protease inhibitor). The lysate may then be partially purified by centrifugation to remove debris, and at least a portion of the supernatant, comprising solubilized FTP, may be applied to the SEC column.

In another non-limiting embodiment, an expression construct encoding a FTP may be transformed into a competent bacterial cell, the cell cultured in liquid growth media to produce a population of cells expressing FTP, and the FTP-expressing cells may be collected and lysed in lysozyme-containing sonication buffer by sonication. The resulting sonicated sample may then be centrifuged to remove debris and to collect a membrane-containing pellet. The membrane pellet may then be solubilized in a buffer comprising a detergent, and then centrifuged again to produce a supernatant, at least a portion of which may be applied to an SEC column.

5.3 Elution Profile Assessment

The system described in section 5.2 may be used to produce an elution profile of the FTP, based on the fluorescence manifested by successive eluent from the SEC column. According to the invention, an elution profile characterized by a substantially symmetrical, single peak indicates that the test protein is monodisperse and likely to crystallize, whereas an elution profile characterized by multiple peaks, or an asymmetric peak, indicates that the test protein is unlikely to crystallize.

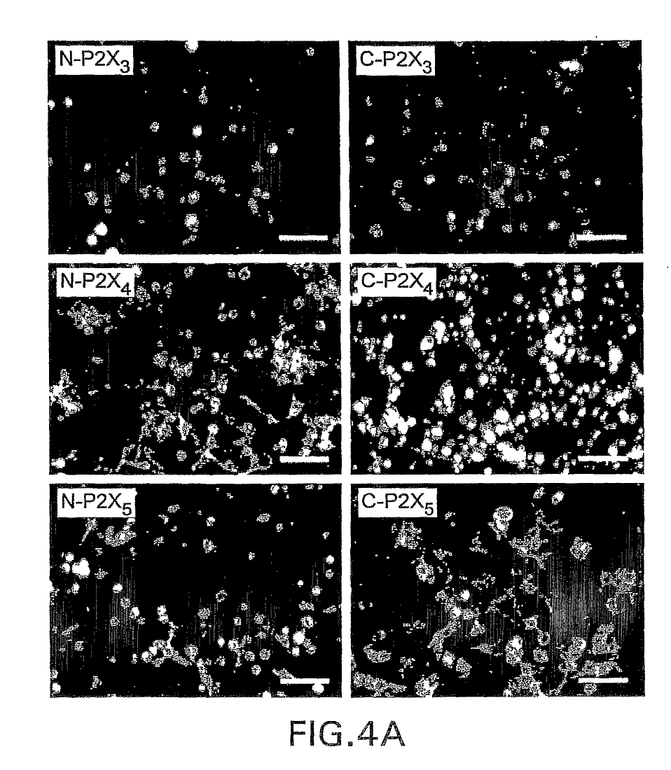

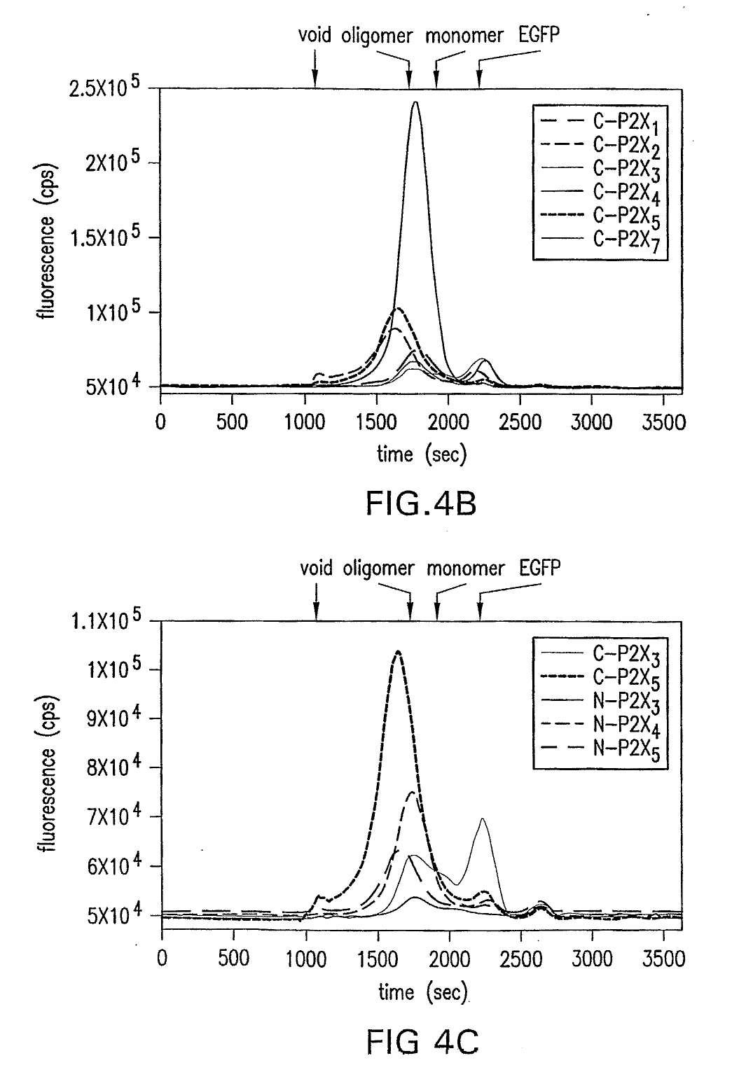

The elution profile comprises a sequence of related values, wherein a first sequence of values represent time, total eluent volume, and/or fraction number, and a second corresponding sequence of values represent fluorescence as measured by the fluorimeter. In preferred non-limiting embodiments of the invention, the first and second sequences of related values are represented by Cartesian coordinates that may be depicted as a two-dimensional graph, wherein one axis represents time, total eluent volume, and/or fraction number and a second axis perpendicular to the first axis represents fluorescence. As a specific non-limiting example, and as depicted in FIGS. 4B and 4C, the abscissa may represent time and the ordinate may represent fluorescence, and a tracing of fluorescence over time is the elution profile.

A “peak,” as that term is used herein, refers to the elution profile of fluorescence relative to time, total eluent volume, and/or fraction number. A peak is defined as having a single local maximum value. A peak may have a shoulder, which hypothetically could reflect the presence of a second peak, but a shoulder will not be considered a second peak herein unless it includes a local maximum bounded on both sides by lower fluorescence values.

A “substantially symmetrical” peak is defined as having substantially the same areas on either side of the local maximum that defines the peak, over intervals in time, total eluent volume, and/or fraction number that are substantially equal on either side of the value of time, total eluent volume, and/or fraction number that corresponds to the local maximum that defines the peak. As a non-limiting example, in a tracing of fluorescence over time, where fluorescence is represented on the ordinate axis and time on the abscissa of a Cartesian graph, and where a local maximum occurs at 30 minutes, the peak is considered symmetric if the areas of the peak are substantially equal on either side of a perpendicular drawn through the local maximum to the abscissa, bounded by the same interval on either side of the perpendicular (e.g., a two-minute span, from 28 minutes to 32 minutes). Preferably, the interval over which peak symmetry is measured does not contain any local minimums (a minimum value of fluorescence bounded on either side by greater values). “Substantially” equal means equal to within 20 percent, preferably to within 15 percent, and more preferably to within 10 percent. The peak areas within the interval being evaluated may be measured using standard computer software, may be measured by visual assessment, or may be measured by cutting out and weighing the areas to be compared. Because malting the interval too narrow may lead to an incorrect determination of asymmetry, in non-limiting embodiments of the invention it is desirable that the area within the interval to be evaluated (that is, on both sides of the local maximum) constitute at least about 60 percent, preferably at least about 70 percent, more preferably at least about 80 percent, of the total peak area.

A peak that is not substantially symmetrical is asymmetrical.

A peak in fluorescence eluting at the void volume may represent destabilized FTP. Where the peal, eluting at the void volume corresponds to more than about 30 percent of total FTP, then the FTP is considered unstable under the conditions used.

For instances where visual inspection of an elution profile is insufficient to determine if a peak is symmetrical, the profile may be fit to Gaussian functions to determine the proteins monodispersity and suitability for crystallization. Elution profiles which are fitted with increasing numbers of Gaussian functions are more likely to contain heterogeneous protein aggregates, and therefore suitable for crystallization (Barth et al., 1994).

5.4 Detergent Screening

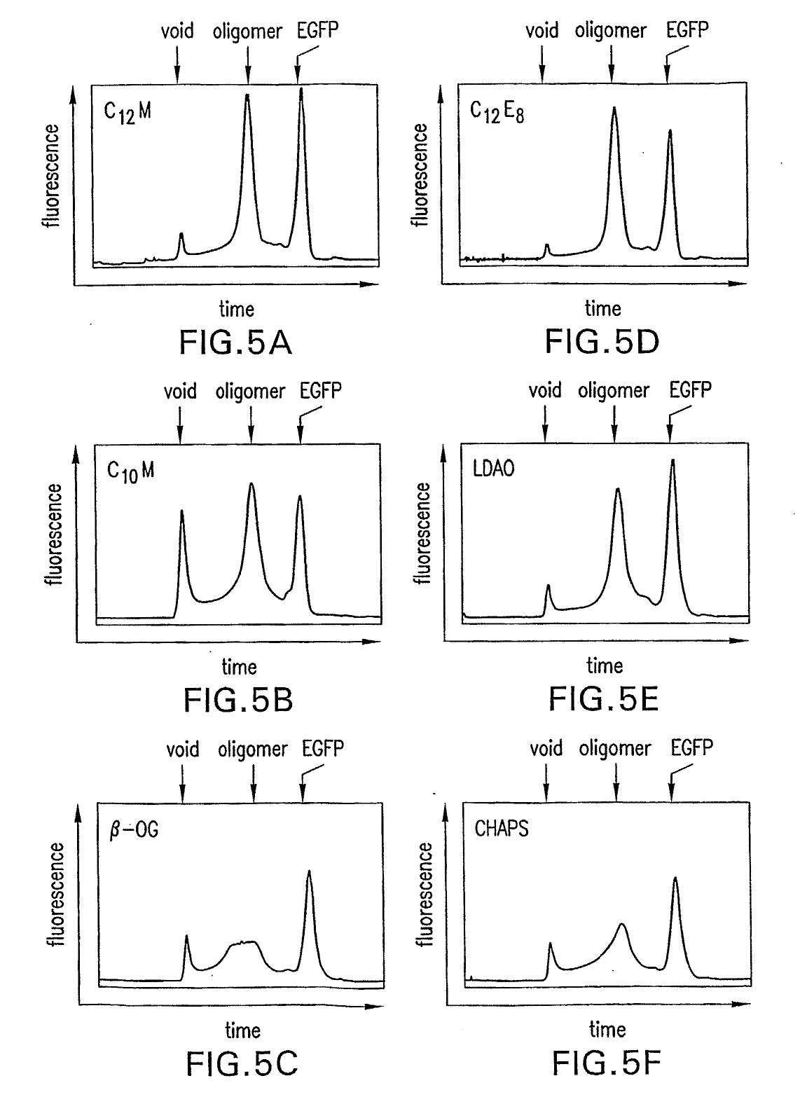

The present invention may be used to evaluate whether a particular detergent is suitable for solubilizing a protein in a monodisperse state for crystallization purposes. According to the invention, an SEC/fluorescence detection system as described in Section 5.3 may be used to determine whether a particular detergent, used to solubilize a FTP, produces a substantially symmetric peak in the elution profile of the FTP. A number of different detergents may be evaluated to identify the detergent which results in the FTP elution profile having greater symmetry (see Section 6, below, and FIG. 5A-F). A FTP that does not assume a stable monodisperse form when solubilized by a detergent may elute at the void volume after SEC, indicating that the detergent may not be suitable for use in crystallization studies of the test protein.

5.5 Uses of the Invention

The present invention may be used to identify and/or select a test protein that is more likely than others to form crystals, where the crystals may then be used in X-ray diffraction studies to determine the three-dimensional structure of the test protein. In preferred non-limiting embodiments of the invention, after determining that the elution profile of an FTP is substantially symmetrical, purified test protein may be produced, either de novo or by removing the fluorescent protein from the FTP (e.g., via an engineered enzyme cleavage site), and then subjected to conditions that favor crystallization, so as to produce crystals that may be used in diffraction studies or that corroborate that the test protein is crystalizable.

A crystal structure determined using a test protein identified as likely to form crystals according to the invention may be used to identify or design molecules that interact with and/or modify the test protein. For example, where the test protein is a membrane ion transporter protein, the crystal structure may be used to identify molecules that modulate ion transport for the protein, and that potentially could be used to treat diseases and disorders associated with ion transport.

In additional embodiments, an FTP-encoding expression construct may be introduced into a bacterial or eukaryotic cell to evaluate the level of expression attainable and to evaluate cellular compartmentalization of the FTP. As one non-limiting example, if a FTP-encoding expression construct is introduced into a bacterial cell but is poorly expressed, then it may be more difficult to produce sufficient quantities of the FTP for further study, relative to a FTP expressed at high levels. In eukaryotic cells transfected with a FTP-encoding expression construct, the sub-cellular localization of the FTP may be determined using epifluorescent microscopy, which may give insight into the structural similarity between the test protein and its corresponding FTP.

### 6. EXAMPLE

### Fluorescence-Detection Size Exclusion Chromatography for Precrystallization Screening of Integral Membrane Proteins

6.1 Experimental Procedures

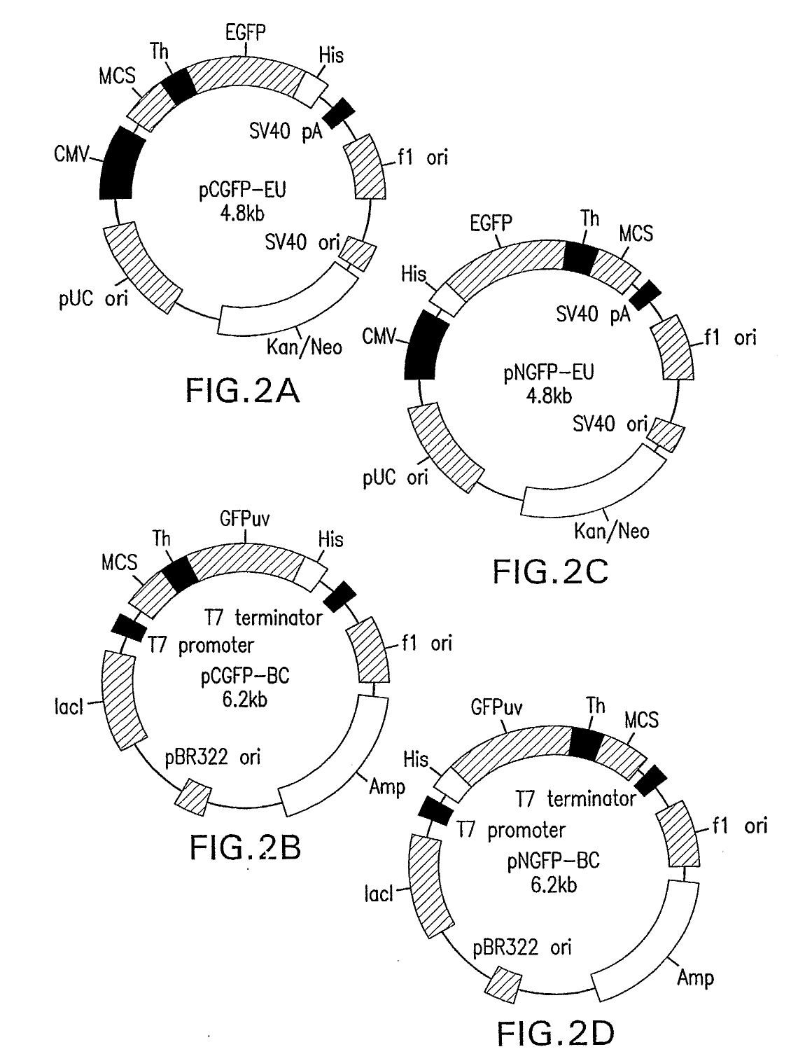

GFP-fusion Vector Construction. (see FIG. 2A-D) The eukaryotic GFP-fusion vectors (PCGFP-EU and pNGFP-EU) were created using standard molecular biology techniques starting with the pEGFP-C1 vector obtained from Clontech. Briefly, the original multiple cloning site (MCS) was replaced with either an octahistidine coding sequence (His8) for pCGFP-EU or with a thrombin recognition site coding sequence (TRS) followed by a new MCS (XhoI-HindIII-EcoRI-PstI-SalI) for pNGFP-EU. The NheI and NotI sites at the 5′ side of the EGFP sequence were used to introduce either the new MCS and TRS for pCGFP-EU or the His 8 sequence for pNGFP-EU. To minimize EGFP dimerization, Ala 206 was mutated to a lysine residue by PCR.

The N-terminal bacterial GFP-fusion vector (pNGF-BC) was created by inserting GFPuv into the pET22c (Novagen) vector, together with the His8 and TRS described above. The original GFPuv was obtained from a plasmid containing GFPuv and was modified by PCR to (i) change Ala 206 to a lysine, ii) remove XhoI, BamHI, HindIII, and NcoI sites, and (iii) add a poly-asparagine linker at the 3′ end (GFPuv-β). SpeI site in the pET22c vector was knocked out by PCR and a His8-TRS was inserted into the pET22c vector between the NdeI and NcoI sites (pET22c-β). Subsequently, the MCS in pET22c-β between the BamHI and XhoI sites was replaced by a pair of synthetic oligos in order to introduce a stop codon after the XhoI site (pET22c-γ). Finally, GFPuv-β was inserted into pET22c-γ between AgeI and SpeI site to produce pNGFP-BC.

The C-terminal bacterial GFP-fusion vector (pCGFP-BC) was created using GFPuv-β and pET25b (Novagen). Briefly, a pelB leader peptide sequence was removed from pET25b by a pair of synthetic oligos (pET25b-β). TRS and His8 coding sequences were added to GFPuv-β at its 5′ and 3′ ends, respectively, by PCR, and inserted between the XhoI and NheI sites of pET25b-β. In order to make the MCS of pCGFP-BC compatible with that of pNGFP-BC, a NcoI site was knocked out and the NdeI site was converted to a new NcoI site by PCR. The resulting MCS includes NcoI, BamHI, EcoRI, SacI, SalI, HindIII, NotI, and XhoI sites. Finally, the first Met in GFPuv was mutated to Val to minimize internal translation initiation.

FSEC for Eukaryotic Cells. HEK293 cells were cultured to ˜90% confluency in a 6-well plate (Corning) and transiently transfected with the specific expression constructs (1 μg/well) using Lipofectamine 2000 (3-5 μl, Invitrogen) as instructed by the manufacturer. After 24-48 hours of incubation, the cells were harvested by gentle pipetting and washed with PBS and resuspended in 500 μl of solubilization buffer (PBS pH 8.0, 20 mM C12M, and 1 μl of protease inhibitor cocktail set III (Calbiochem)). The resulting suspension was rotated for 1 hour at 4° C. followed by centrifugation at 66,000×g for 40 min. A fraction of the supernatant (200 μl) was loaded onto a Superose 6 column (10/30, Amersham Biosciences), run at a flow rate of 0.5 ml/ml, and equilibrated in SEC buffer (20 mM Tris pH 8.0, 150 mM NaCl, 1 mM EDTA, and 1 mM C12M). The eluent from the SEC column was passed through a fluorometer fitted with a flow cell. The fluorimeter settings were as follows: band pass: 5 nm/5 nm; excitation: 488 nm; emission: 507 nm; time increment: 1 sec; integration time: 1 sec; and recording time: 3000-3600 sec. Calibration using known quantities of GFP demonstrated that 1-10 ng of GFP could be readily detected.

FSEC for Bacterial Cells. The desired expression vector was transformed into BL21(DE3) pLysS competent cells using standard methods, and the resulting cells were plated onto LB agar plates supplemented with ampicillin and chloramphenicol. Following incubation for ˜24 hr at 37° C., a single colony was picked and used to inoculate 10 ml of LB medium containing 50 μg/ml ampicillin and 34 μg/ml chloramphenicol. Cells were cultured in a shaker at 37° C., when the OD600 reached ˜0.6, expression was induced by the addition of 1 mM IPTG and the cells were cultured for an additional 3 hours. The cells were collected by centrifugation, resuspended in 500 μl of sonication buffer (50 mM Tris-HCl, pH 8.0, 190 mM NaCl, 10 mM KCl, 15 mM EDTA, 0.01 mg/ml lysozyme and 100 μM PMSF), and disrupted by sonication on ice. Sonication was repeated 4 times with 1 min intervals using VirSonic475 (Virtis) in which each cycle was programmed as follows: sonication time: 1 sec; interval: 1 sec; total sonication time: 10 sec. The sonicated sample was first centrifuged at 23,000×g for 15 min to pellet unbroken cells, and then the membranes were collected by a second centrifugation at 200,000×g for 20 min. The membrane pellet (˜5-20 μg) was solubilized with 500 μl of solubilization buffer 1 (50 mM Tris-HCl, pH 8.0, 190 mM NaCl, 10 mM KCl, 15 mM EDTA, 100 μM PMSF, and 40 mM C12M) and gently mixed at 4° C. for at least 1 hr, followed by centrifugation at 66,000×g for 20 min. A fraction of the resulting supernatant (200 μl) was loaded onto a Superose 6 column equilibrated in running buffer (20 mM Tris-HCl, pH 8.0, 190 mM NaCl, 10 mM KCl, 1 mM C12M) and run at the flow rate of 0.5 ml/min. The eluent was detected by a fluorimeter as described above, with the only difference being the excitation wavelength (395 nm) and emission wavelength settings (507 nm).

Detergent Stability Screening of an Eulcarvotic Membrane Protein J. Eukaryotic membrane protein J was expressed in Sf9 cells by baculovirus infection for 72 hours at 27° C. using standard methods. The baculovirus was created from pFastBac (Invitrogen) in which the entire coding sequence of C-terminally tagged protein J from pCGFP-EU vector was inserted between the BamHI and HindIII sites. Cells from 1 ml of culture were collected by centrifugation and solubilized in 300 μl of PBS (Cellgro) containing one of the following detergents, where the final detergent concentration is given in parentheses: C12M (20 mM), C10M (20 mM), β-OG (250 mM), C12E8 (20 mM), LDAO (20 mM), and CHAPS (125 mM). Solubilization was carried out for 1 hour at 4° C. with gentle rotation. After centrifugation at 66,000×g for 40 min, 200 μl of the soluble fraction was loaded onto a Superose 6 10/30 column and FSEC was carried out as described above.

Gaussian Peak Fitting. Fluorescence values between 1000 seconds and 2500 seconds on the FSEC traces of C-Glt_{Ph} and N-Glt_{Ph} were imported into PeakFit software (Sea Solve Software, Inc). Initial peak detection and fitting was done by the residual method and the Gaussian functions were further fitted to the original peaks using least square minimization algorithm with iteration cycles of 61 (C-Glt_{PH}) and 34 (N-Glt_{PH}), respectively. The resulting Gaussian peaks were validated by r² coefficient of determinant, degree of freedom adjusted coefficient of determination, fit standard error, and F-value.

6.2 Results and Discussion

Covalent GFP Fusions. The precrystallization screening methodology has two facets. The first involves a series of expression vectors in which the target gene is covalently fused to green fluorescent protein (GFP). Fused to the terminus of GFP is a poly histidine tag for affinity purification and inserted between the target protein and GFP is a thrombin site for proteolytic cleavage of the target protein from GFP, as illustrated in FIG. 2A-D. Enhanced Green Fluorescent Protein (EGFP) was chosen for eukaryotic expression and GFPuv was chosen for bacterial expression because of: i) high stability in each expression system, ii) stronger fluorescence signals compared to the wild-type counterparts, and iii) optimized codon-usage for each expression system (Crameri et al., 1996; Haas et al., 1996; Heim et al., 1995). To reduce dimer formation through GFP-GFP interaction, alanine 206 in both GFP variants were mutated to lysine (Zacharias et al., 2002). In each expression vector, a multiple cloning site (MCS) is located either at the 5′ or 3′ side of the GFP coding sequence to tag GFP either at the N- or C-terminus of a protein of interest. Because identical MCSs are used for both N- and C-terminal GFP-fusion vectors for each expression system, one can quickly screen a tagging position of GFP using the same PCR product.

Covalently-fused GFP instantaneously allows one to visually inspect subcellular localization of proteins in eukaryotic cells and to determine protein expression in bacterial cells by batch fluorescence. Moreover, polyhistidine and thrombin sites in the GFP fusion vectors allow one to purify and characterize the proteins from a small amount of cells, talking advantage of the sensitive fluorescence from GFP. These features profoundly benefit precrystallization screening of integral membrane proteins whose expression levels are usually lower significantly than those of soluble proteins.

Fluorescence-detection Size Exclusion Chromatography. Another aspect of the precrystallization screening methodology is a fluorometry-coupled chromatography system fitted with a SEC column. With this setup, one can monitor the elution of GFP-fusion proteins in the context of whole cell lysates or solubilized crude membranes, as shown in FIG. 3. This method is referred to as fluorescence-detection SEC (FSEC). In theory, one can carry out FSEC precrystallization screening on both water-soluble and membrane proteins.

Typically, crude membranes from bacterial cells or intact HEK 293 cells are solubilized in a detergent-containing solution. This crude fraction is then directly applied to a SEC column equilibrated in a detergent-containing solution, and the column is connected to a fluorometer fitted with a flow cell (FIG. 3B). SEC is a powerful screening method because the peak areas and profiles provide information on (1) the expression level, (2) the degree of monodispersity, and (3) the approximate molecular mass of the fusion protein. Because FSEC exploits the unique fluorescent signal of GFP, neither protein purification nor large scale culture is required since a typical fluorimeter can detect ˜10 ng of GFP. One helpful addition includes a UV detector for calibrating a SEC column by absorbance at 280 nm using known standard proteins. A second beneficial addition includes a fraction collector so that each peak fraction can be analyzed by a series of biochemical assays such as Western blot, enzymatic activity assays, or ligand binding experiments.

Subtype Screening of P2X Receptors by FSEC. P2X receptors are eukaryotic integral membrane proteins that form ion channels gated by ATP (Khakh, 2001; North, 2002). There are seven P2X receptor subtypes (P2X1-7), and all subtypes except P2X6 form functional channels when expressed in HEK 293 cells. To determine whether any of the rat P2X receptor subtypes would be suitable for crystallization trials, precrystallization screening was performed as described above. PCR products of P2X1-5,7 genes were subcloned into either pCGFP-EU or pNGFP-EU, and the resulting plasmids were transfected into HEK 293 cells. Two days after transfection, the subcellular localization of P2X receptors were checked by fluorescence microscopy (FIG. 4A). According to the images, C-terminally tagged P2X receptors (C-P2Xs) expressed more robustly compared to N-terminally tagged variants. In the case of P2×3, both the amino and carboxy terminal variants were predominately localized to the cytoplasm, while in the case of P2×4, C-P2X4 was found in intracellular puncta and N-P2X4 was found primarily on the cell surface. This unique localization pattern of C-terminally tagged P2X receptors were consistent to what has been reported previously (Bobanovic et al., 2002).

To quantitatively evaluate the expression level and degree of monodispersity, the amino and carboxy terminal GFP fusion P2X constructs were expressed on a small scale in transiently transfected HEK293 cells, using 3.5 cm dishes for each construct. The cells were solubilized in n-dodecyl β-D-maltoside (C12M) and the resulting supernatant was analyzed by FSEC. As shown in FIG. 4B, the fluorescence peak associated with the C-P2X4 construct was much larger than the peaks from the other constructs, thus confirming the tentative observation that C-P2X4 expressed at a higher level than the other constructs. Notably, the fluorescence peak was nearly symmetric, the elution position of the peak was suggestive of an oligomer, and there was only a small peak at the void volume of the column. The overall expression level was estimated to ˜1 μg/1×10⁶ cells using a standard correlation curve between concentrations of recombinant EGFP and their fluorescence. Taken together, these observations indicated that the C-P2X4 construct formed a defined oligomer and that the C-P2X4 protein expressed fairly high but was not prone to the formation of large aggregates.

The other fusion proteins, C-P2X5, C-P2X7, N-CP2X4, and N-P2X5 had reasonably symmetrical peak shapes but they all expressed at much lower levels (FIGS. 4B and 4C). The C-P2X2 construct gave rise to a small but significant peak at the void volume, suggesting that it had a tendency to aggregate. Interestingly, the C-P2X3 supernatant contained a substantial amount of free EGFP (FIG. 4C), which was consistent with the observations from fluorescence microscopy (FIG. 4A). Overall, inspection of transfected cells by epi-fluorescence microscopy and the analysis of solubilized cells by FSEC lead to the discovery of a P2X receptor subtype and construct that expressed at the highest level and that yielded a symmetrical peak on a SEC column.

Detergent Screening by FSEC. Successful crystallization of a membrane protein is often critically dependent upon the detergent and, in many cases, the most well-ordered crystals are formed in the presence of that detergent which forms the smallest micelles but which nevertheless maintains the protein in a monodisperse and stable state. The traditional approach to screening detergents involves purification of the membrane protein of interest, exchange of the protein into a panel of detergents, and subsequent evaluation of the degree of monodispersity and stability. In contrast, FSEC may be used to determine the degree of monodispersity and stability of the target protein, without any purification, using whole cell lysates.

A typical example of detergent screening by FSEC using protein J is provided. Protein J is an eukaryotic, integral membrane protein that is a member of the ENaC/MEC family of ion channels (Kellenberger and Schild, 2002). It was expressed in Sf9 insect cells as a carboxy terminal fusion with GFP (C-J) by recombinant baculovirus infection. The baculovirus DNA was created by site specific transposition in E. coli cells with a plasmid containing the entire coding region of the fusion protein. C-J expressing Sf9 cells were subsequently solubilized in 6 detergents (C12M, n-decyl-β-D-maltoside (C10M), n-octyl-β-D-glucoside (β-OG), octaethylene glycol monododecylether (C12E8), lauryl dimethylamine-N-oxide (LDAO), and CHAPS)), following high speed centrifugation, the supernatants were analyzed by FSEC using a column equilibrated in C12M. As shown in FIG. 5, the peak profiles from the solubilized samples, except those in β-OG and CHAPS, were sharp and symmetrical, suggesting that the protein was monodisperse in these detergents. Furthermore, in C12M, C12E8, and LDAO only a small fraction of the protein migrated in the void volume of the column, thereby indicating that the protein did not tend to aggregate under these conditions.

When C-J was solubilized in C10M, however, a significant fraction eluted in the void volume despite the fact that the major peak was still sharp and symmetrical. This observation suggests that although protein J could be solubilized in C10M without disruption of its native association state, it was only marginally stable in C10M. In fact, when the peak fraction was re-analyzed by FSEC after 3 days at 4° C., most of the protein eluted in the void volume. When protein J was analyzed following solubilization in β-OG and CHAPS, the major fluorescent peaks were broad and asymmetric, indicating that these two detergents were not suitable for maintaining protein J in a stable, monodisperse state. In conclusion, these few studies suggested that C12M, C12E8 and LDAO are the most promising detergents for purification and crystallization of protein J.

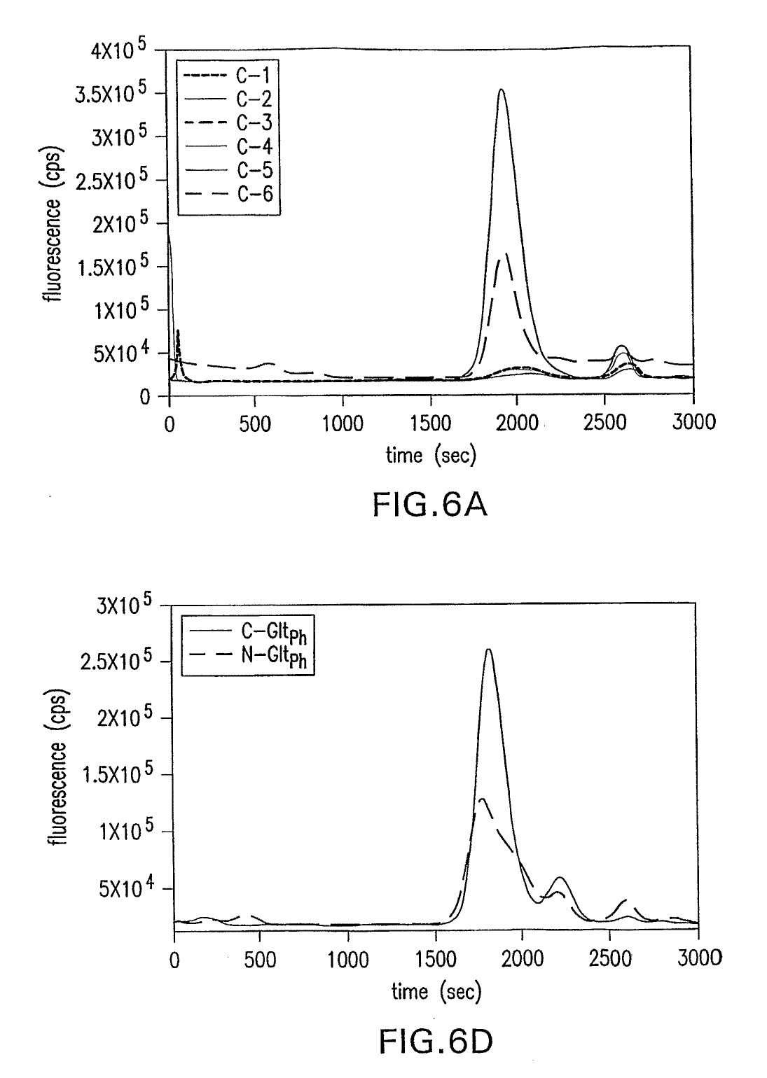

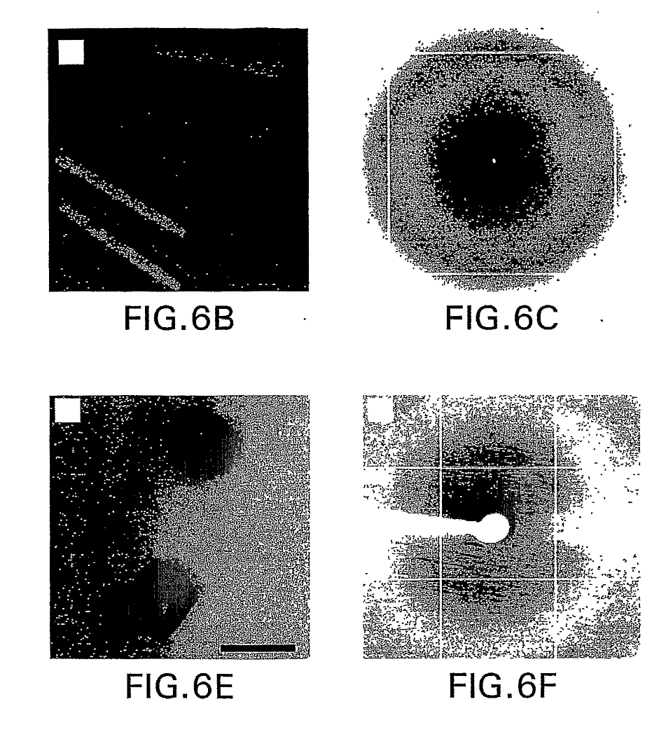

Precrystallization Screening of a Bacterial Membrane Protein by FSEC. To obtain crystals of a bacterial integral membrane protein, genes from 6 different organisms (gene 1-6) were subcloned into either pCGFP-BC (C-1, C-2, . . . C-6) or pNGFP-BC (N-1, N-2, . . . N-6) and screened by FSEC. As shown in FIG. 6A, the expression levels and the degree of monodispersity of the carboxy terminal GFP fusions varied substantially. Moreover, the expression of constructs C-2 and C-6 was much greater than the expression of C-1, C-3 and C-5. Interestingly, the proteins that were more abundantly expressed yielded more symmetric peaks whereas those that were poorly expressed gave less symmetric peaks, indicative of aggregation or heterogeneity in subunit stoichiometry. Although the calculated molecular masses of the fusion proteins are within 10%, proteins C-1 and C-3 eluted significantly later than the other proteins. One explanation for this behavior is that proteins C-1 and C-3 were binding to the resin, which may have been due to misfolding or partial unfolding. The FSEC traces for all the amino terminal fusions showed similar profiles except that N-2 peak was smaller than N-6 peak. This suggested that gene 2 product was more stable with C-terminal tag while gene 6 did not have preference for the tagging position. On the basis of the FSEC screening, the target proteins of the C-2 and C-6 constructs were subjected to crystallization trials. In order to simplify the purification procedure, these target proteins were expressed and purified on a large scale in the absence of GFP. The target protein from the C-6 construct crystallized readily, yielding crystals that diffracted beyond 2 Å resolution, as illustrated in FIGS. 6B and 6C.

FSEC Profiles of GltPh. To understand why amino-terminally-tagged variants of the trimeric glutamate transporter homolog from Pyrococcus horikoshi (GltPh) (Yernool et al., 2004) did not express as well as carboxy-terminally-tagged constructs and did not yield crystals, the GltPh gene was cloned into the pCGFP-BC and pNGFP-BC E. coli expression vectors, yielding the C-GltPh and N-GltPh constructs. Analysis of crude solubilized membranes by FSEC (FIG. 6D) showed that while C-GltPh had a narrow and symmetric peak, N-GltPh yielded a smaller and asymmetric peak, suggestive of heterogeneity in subunit stoichiometry and/or incomplete assembly. These results are consistent with previous observations made prior to the development of FSEC technology. Because C-terminally tagged GltPh formed diffraction quality crystals (FIGS. 6E and 6F) and amino terminally tagged variants did not crystallize, even after the purification tags were proteolytically removed, the FSEC results provide an explanation for the poor crystallization behavior of the amino terminally tagged constructs.

At the molecular level, inspection of the GltPh crystal structure does show that the carboxy terminus is projecting away from the protein (Yernool et al., 2004), so it appears that the protein can accommodate a carboxy terminal tag. A molecular understanding of the difficulties encountered with amino terminal tags is less clear, in part because the first few residues of the protein can not be reliably positioned in electron density. However, there are electron density features that suggest that the amino terminus makes contact with the protein core, and this may be why amino terminal fusions are not tolerated. Nevertheless, the studies of the amino and carboxy terminal fusions of GltPh highlight the importance of screening both amino and carboxy terminal fusions, and they emphasize how FSEC precrystallization analysis can provide important information, rapidly and easily.

Gaussian Peak Fitting of Elution Profiles. When visual inspection of FSEC elution profiles is inadequate, the expression level and degree of monodispersity of the test protein can be estimated by fitting the peak shapes to Gaussian functions. Depending on the number of functions necessary to fit any particular peak, the abundance of species composing that peak can be estimated (Barth et al., 1994). If a peak from a FSEC trace requires multiple Gaussians to achieve a reasonable fit, then it is likely that the target protein is heterogeneous in aggregation or association state and not suitable for crystallization trials.

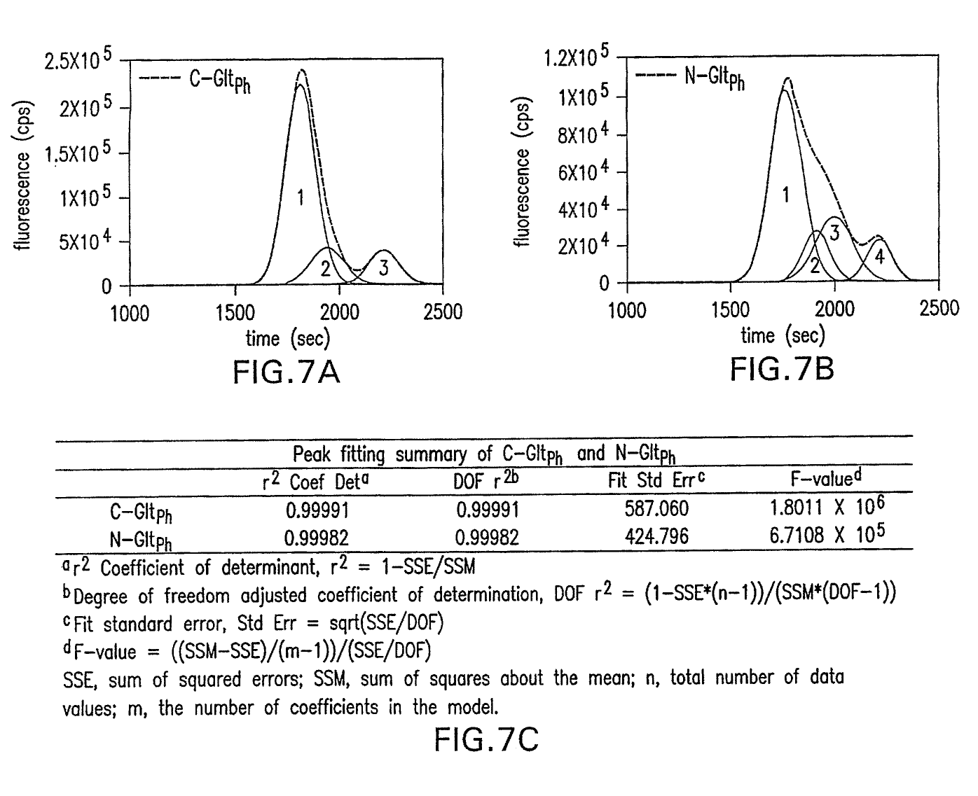

To quantitatively compare the peak shapes of C-Glt_{Ph} and N-Glt_{Ph}, the FSEC traces of these proteins were fit with multiple Gaussian functions. As shown in FIG. 7, the C-Glt_{Ph}, trace was adequately fit by three Gaussian functions (peaks 1-3) while the N-Glt_{Ph} trace required four Gaussian functions (peaks 1-4). In addition to requiring an additional function, the N-Glt_{Ph} construct has a substantial third peak that likely corresponds to monomeric subunits of the transporter. Gaussian fitting of the FSEC peaks therefore shows that N-Glt_{Ph} samples exhibited substantially more heterogeneity than its C-Glt_{Ph} counterpart, and therefore less suitable for crystallization.

In conclusion, it has been shown that large-scale protein expression and purification can be minimized by fluorescence-detection size exclusion chromatography (FSEC), a rapid precrystallization screening method in which monodispersity and stability of the target protein are characterized with only nanogram quantities of unpurified protein. In this method, the target protein is covalently fused to GFP and the resulting unpurified fusion protein is analyzed by SEC. Although GFP fusion proteins have been used previously to monitor membrane protein expression in bacteria and to establish mammalian cell lines producing more proteins, it is novel to combine covalent GFP fusion and SEC techniques to analyze monodispersity and stability of the fusion protein for protein crystallization (Drew et al., 2001; Mancia et al., 2004).

In this report, the advantage and significance of covalent GFP fusion proteins and FSEC precrystallization screening were demonstrated in the examples with prokaryotic and eukcaryotic membrane proteins. In these experiments, small amounts of the unpurified target membrane proteins were rapidly and easily evaluated for the localization and expression level, degree of monodispersity, approximate molecular mass, and stability in detergents. The utility of this approach is dramatically illustrated by its successful application to the described prokaryotic membrane protein, which eventually formed crystals that diffracted beyond 2.0 Å. Therefore, FSEC precrystallization screening is an efficacious protein characterization method which will significantly reduce the time and resource required for membrane protein crystallization.

### 7. REFERENCES CITED

Aschrafi, A., Sadtler, S., Niculescu, C., Rettinger, J., and Schmalzing, G. (2004). Trimeric architecture of homomeric P2X2 and heteromeric P2X1+2 RECEPTOR SUBTYPES. J Mol Biol 342, 333-343.
Bardoni, R., Goldstein, P. A., Lee, C. J., Gu, J. G., and MacDermott, A. B. (1997). ATP P2X receptors mediate fast synaptic transmission in the dorsal horn of the rat spinal cord. J Neurosci 17, 5297-5304.
Barth, H. G., Boyes, B. E., and Jackson, C. (1994). Size exclusion chromatography. Anal Chem 66, 595R-620R.
Blundell, T. L., Jhoti, H., and Abell, C. (2002). High-throughput crystallography for lead discovery in drug design. Nat Rev Drug Discov 1, 45-54.
Bobanovic, L. K., Royle, S. J., and Murrell-Lagnado, R. D. (2002). P2X receptor trafficking in neurons is subunit specific. J Neurosci 22, 4814-4824.
Cockayne, D. A., Hamilton, S. G., Zhu, Q. M., Dunn, P. M., Zhong, Y., Novakovic, S., Malmberg, A. B., Cain, G., Berson, A., Kassotakis, L., Hedley, L., Lachnit, W. G., Burnstock, G., McMalion, S. B., and Ford, A. P. (2000). Urinary bladder hyporeflexia and reduced pain-related behaviour in P2×3-deficient mice. Nature 407, 1011-1015.
Cook, S. P., Vulchanova, L., Hargreaves, K M., Elde, R., and MaCleskey, E. W. (1997). Distinct ATP receptors on pain-sensing and stretch-sensing neurons. Nature 387, 505-508.
Crameri, A., Whitehorn, E. A., Tate, E., and Stemmer, W. P. (1996). Improved green fluorescent protein by molecular evolution using DNA shuffling. Nat Biotechnol 14, 315-319.
Drew, D. E., von Heijne, G., Nordlund, P., and de Gier, J. W. (2001). Green fluorescent protein as an indicator to monitor membrane protein overexpression in Escherichia coli. FEBS Left 507, 220-224.
Eniion, S., Hagan, S., and Evans, R. J. (200). The role of positively charged amino acids in ATP recognition by human P2X(1) receptors J Biol Chem 275, 29361-29367.
Gu, J. G., and MacDermott, A. B. (1997). Activation of ATP P2X receptors elicits glutamate release from sensory neuron synapses. Nature 389, 749-753.
Haas, J., Park, E. C., and Seed, B. (1996). Codon usage limitation in the expression of HIV-1 envelope glycoprotein. Curr Biol 6, 315-324.
Heim, R., Cubitt, A. B., and Tsien, R. Y. (1995). Improved green fluorescence. Nature 373, 663-664.
Hendrickson, W. A. (2000). Synchrotron crystallography. Trends Biochem Sci 25, 637-643.
Illes, P., Ribeiro, J. A. (2004). Neuronal P2 receptors of the central nervous system. Curr Top Med Chem 4, 831-838.
Inscho, E. W., Cook, A. K., Imig, J. D., Vial, C., and Evans, R. J. (2004). Renal autoregulation in P2X1 knockout mice. Acta Physiol Scand 181, 445-453.
Jiang, L. H., Rassendren, F., Surprenant, A., and North, R. A. (2000). Identification of amino acid residues contributing to the ATP-binding site of a purinergic P2X receptor. J Biol Chem 275, 34190-34196.
Kaldor, S. W., Kalish, V. J., Davies, J. F., 2nd, Shetty, B. V., Fritz, J. E., Appelt, K., Burgess, J. A., Campanale, K. M., Chirgadze, N.Y., Clawson, D. K., et al. (1997). Viracept (nelfinavir mesylate, AG1343): a potent, orally bioavailable inhibitor of HIV-1 protease. J Med Chem 40, 3979-3985.
Kellenberger, S., and Schild, L. (2002). Epithelial sodium channel/degenerin family of ion channels: A variety of functions for a shared structure. Physiol Rev 82, 735-767.
Khakh, B. S. (2001). Molecular physiology of P2X receptors and ATP signalling at synapses. Nat Rev Neurosci 2, 165-174.
Kuhn, P., Wilson, K., Patch, M. G., and Stevens, R. C. (2002). The genesis of high-throughput structure-based drug discovery using protein crystallography. Curr Opin Chem Biol 6, 704-710.
Lasbasi, J. M., Petrushova, N., Donovan. C., McCurdy, S., Lira, P., Payette, M. M., Brissette, W., Wicks, J. R., Audoly, L., and Gabel, C. A. (2002). Absence of the P2X7 receptor alters leukocyte function and attenuates an inflammatory response. J Immunol 168, 6436-6445.
Mancia, F., Patel, S. D., Rajala, M. W., Scherer, P. E., Nemes, A., Schieren, I., Hendrickson, W. A., and Shapiro, L. (2004). Optimization of protein production in mammalian cells with a coexpressed fluorescent marker. Structure (Camb) 12, 1355-1360.
Mulryan, K., Gitterman, D. P., Lewis, C. J., Vial, C., Leckie, B. J., Cobb, A. L., Brown, J. E., Conley, E. C., Buell, G., Pritchard, C. A., and Evans, R. J. (2000). Reduced vas deferens contraction and male fertility in mice lacking P2X1 receptors. Nature 403, 86-89.
Nakatsuka, T., and Gu, J. G. (2001). ATP P2X receptors-mediated enhancement of glutamate release and evoked EPSCs in dorsal horn neurons of the rat spinal cord. J Neurosci 21, 6522-6531.
Nicke, A., Baumert, H. G., Rettinger, J., Eichele, A., Lambrecht, G., Mutschler, E., and Schmalzing, G. (1998). P2X1 and P2X3 receptors form stable trimers: a novel structural motif of ligand-gated ion channels. Embo J 17, 3016-3028.
Nicke, A., Rettinger, J., and Schmalzing, G. (2003). Monomeric and dimeric byproducts are the principal functional elements of higher order P2X1 concatamers. Mol Pharmacol 63, 243-252.
Nishida, M., and MacKinnon, R. (2002). Structural basis of inward rectification: cytoplasmic pore of the G protein-gated inward rectifier GIRK1 at 1.8 Å resolution. Cell 111, 957-965.
North, R. A. (2002). Molecular physiology of P2X receptors. Physiol Rev 82, 1013-1067.
North, R. A., and Barnard, E. A. (1997). Nucleotide receptors. Curr Opin Neurobiol 7, 346-357.
Papp, L., Vizi, E. S., and Sperlagh, B. (2004). Lack of ATP-evoked GABA and glutamate release in the hippocampus of P2X7 receptor−/− mice. Neuroreport 15, 2387-2391.
Ren, J., Bian, X., DeVries, M., Schnegelsberg, B., Cockayne, D. A., Ford, A. P., and Galligan, J. J. (2003). P2X2 subunits contribute to fast synaptic excitation in myenteric neurons of the mouse small intestine. J Physiol 552, 809-821.
Roberts, J. A., and Evans, R. J. (2005). Mutagenesis studies of conserved proline residues of human P2X receptors for ATP indicate that proline 272 contributes to channel function. J Neurochem 92, 1256-1264.
Rong, W., Gourine, A. V., Cockayne, D. A., Xiang, Z., Ford, A. P., Spyer, K. M., and Burnstock, G. (2003). Pivotal role of nucleotide P2X2 receptor subunit of one of the ATP-gated ion channel mediating ventilatory responses to hypoxia. J Neurosci 23, 11315-11321.
Rubio, M. E., and Soto, F. (2001). Distinct localization of P2X receptors at excitatory postsynaptic specializations. J Neurosci 21, 641-653.
Solle, M., Labasi, J., Perregaux, D. G., Stam, E., Petrushova, N., Koller, B. H., Griffiths, R. J., and Gabel, C. A. (2001). Altered cytokine production in mice lacking P2X(7) receptors. J Biol Chem 276, 125-132.
Souslova, V, Cesare, P., Ding, Y., Akopian, A. N., Stanfa, L., Suzuki, R., Carpenter, K., Dickenson, A., Boyce, S., Hill, R., Nebenuis-Oosthuizen, D., Smith, A. J., Kidd, E. J., and Wood J. N. (2000). Warm-coding deficits and aberrant inflammatory pain in mice lacking P2X3 receptors. Nature 407, 1015-1017.
Tate, C. G. (2001). Overexpression of mammalian integral membrane proteins for structural studies. FEBS Lett 504, 94-98.
Tsuda, M., Shingemoto-Mogami, Y., Koizumi, S., Mizokoshi, A., Kohsaka, S., Salter, M. W., and Inoue, K. (2003). P2X4 receptors induced in spinal microglia gate tactile allodynia after nerve injury. Nature 424, 778-783.
Valera, S., Hussy, N., Evans, R. J., Adami, N., North, R. A., Surprenant, A., and Buell, G. (1994). A new class of ligand-gated ion channel defined by P2X receptor for extracellular ATP. Nature 371, 516-519.
Walian, P., Cross, T. A., and Jap, B. K. (2004). Structural genomics of membrane proteins. Genome Biol 5, 215.
Watano, T., Calvert, J. A., Vial, C., Forsythe, I. D., and Evans, R. J. (2004). P2X receptors subtype-specific modulation and excitatory and inhibitory synaptic inputs in the rat brainstem. J Physiol 558, 745-757.

Wlodawer, A., and Vondrasek, J. (1998). Inhibitors of HIV-1 protease: a major success of structure-assisted drug design. Annu Rev Biophys Biomol Struct 27, 249-284.
Yaar, R., Jones, M. R., Chen, J. F., and Ravid, K. (2005). Animal models for the study of adenosine receptor function. J Cell Physiol 202, 9-20.
Yernool, D., Boudker, O., Jin, Y., and Gouaux, E. (2004). Structure of a glutamate transporter homolog from Pyrococcus horikoshii. Nature 431, 811-818.
Zacharias, D. A., Violin, J. D., Newton, A. C., and Tsien, R. Y. (2002). Partitioning of lipid-modified monomeric GFPs into membrane microdomains of live cells. Science 296, 913-916.
Zambrowicz, B. P., and Sands, A. T. (2003). Knockouts model the 100 best-selling drugs—will they model the next 100? Nat Rev Drug Discov 2, 38-51.
Zhong, Y., Dunn, P. M., Xiang, Z., Bo, X., and Burnstock, G. (1998). Pharmacological and molecular characterization of P2X receptors in rat pelvic ganglion neurons. Br J Pharmacol 125, 771-781.

Various patents, patent applications, and other publications are cited herein, the contents of which are hereby incorporated by reference in their entireties.

What is claimed:

1. A method of determining the likelihood that a test protein will crystallize comprising the steps of:
expressing a fluorescent fusion protein comprising portions of the test protein and a fluorescent protein;
preparing a sample comprising the fusion protein, in solubilized form;
performing size exclusion chromatography on solubilized fusion protein;
detecting the fluorescence of eluent fractions containing the fusion protein; and
determining the elution profile of the fusion protein; and
characterizing the elution profile;
wherein an elution profile characterized by a substantially symmetrical, single peak indicates that the test protein is monodisperse and likely to crystallize, whereas an elution profile characterized by multiple peaks, or an asymmetric peak, indicates that the test protein is unlikely to crystallize, and whereas an elution profile in which the fusion protein elutes at the void volume indicates that the solubilized test protein is unstable.

2. The method of claim 1, wherein the fusion protein further comprises an affinity tag.

3. The method of claim 1, wherein the fusion protein comprises a cleavage site between the fluorescent protein and the test protein.

4. The method of claim 1, wherein fusion protein comprises a fluorescent protein linked to an N-terminal portion of the test protein.

5. The method of claim 4, wherein the fluorescent protein is linked to the N-terminal portion of the test protein by a linker molecule.

6. The method of claim 1, wherein fusion protein comprises a fluorescent protein linked to a C-terminal portion of the test protein.

7. The method of claim 6, wherein the fluorescent protein is linked to the C-terminal portion of the test protein by a linker molecule.

8. The method of claim 5, wherein the linker molecule comprises an enzyme cleavage site.

9. The method of claim 7 wherein the linker molecule comprises an enzyme cleavage site.

10. The method of claim 1, wherein the fusion protein is expressed by introducing, into a host cell, an expression construct comprising nucleic acid encoding the fusion protein operably linked to a promoter.

11. The method of claim 1, further comprising producing a lysate of the host cell and solubilizing the fusion protein in the lysate.
